(19) United States
(12) Patent Application Publication
(10) Pub. No.: US 2013/0273650 A1
(43) Pub. Date: Oct. 17, 2013

(54) VECTORS AND METHODS FOR RECOMBINANT PROTEIN EXPRESSION

(71) Applicant: Xiaoyun Wu, Birmingham, AL (US)

(72) Inventor: Xiaoyun Wu, Birmingham, AL (US)

(73) Assignee: ADV BIOLOGICS ,INC., Birmingham, AL (US)

(21) Appl. No.: 13/812,488

(22) PCT Filed: Jul. 26, 2011
(86) PCT No.: PCT/US2011/045316

§ 371 Date: May 6, 2013

Related U.S. Application Data
(60) Provisional application No. 61/367,661, filed on Jul. 26, 2010.

(51) Int. Cl. C12N 15/85

(52) U.S. Cl. CPC C12N 15/85
USPC 435/354; 435/320.1; 435/358; 435/364; 435/366; 435/369

(57) ABSTRACT

The present invention discloses a series of eukaryotic expression vectors utilizing the reduction of transcription read-through events to create stable and high-yield cell lines for recombinant protein expression. The vectors comprise more than one polyadenylation signal or one or more polyadenylation signals plus other DNA fragment which is known to enhance transcription termination to control the expression level of selection marker, with the configuration to transcribe the minimal level of full-length bicistronic mRNA to express the selection marker, which can be used to create stable cell lines at high expression levels, without the need for drug selection or drug mediated gene amplification.

### CROSS-REFERENCE TO RELATED APPLICATIONS

This application claims the benefit of priority of U.S. Ser. No. 61/367,661, filed Jul. 26, 2010. The entire content and disclosure of the preceding application is incorporated by reference into this application.

### FIELD OF THE INVENTION

The present invention relates to expression of recombinant proteins in eukaryotic cells.

### BACKGROUND OF THE INVENTION

Expression of recombinant DNA in mammalian cells has allowed for the production of multiple, complex, glycosylated proteins (such as monoclonal antibodies) for clinical application. A major effort is often required to create the cell line or lines that stably express such proteins at high levels (over 20 pg protein/cell/day). However, the expression levels in the majority of the stable cell lines are often low (less than 1 pg/day/cell) and unstable (diminish over time and in large scale culture). Therefore, numerous individual clones need to be screened (often on the order of thousands of clones) in order to obtain a few highly expressing clones at 20 pg/cell/day (Levinson and Goeddel, 1990; Andersen and Krummen, 2002; Wurm, 2004).

There are two vector designs which use selection markers to help generate stable cells: in the first, expression of an interested gene and selection marker gene are driven by two separate promoters, while in the second the expression of both genes is driven by a single promoter (bicistronic). In the first design, a strong promoter (such as a CMV promoter) is utilized to drive the expression of the target gene (product) and a weak promoter (such as SV40 promoter) to express the selection marker. Chinese hamster ovary (CHO) DHFR-deficient cell (e.g. DG44) and the amplifiable selection marker dihydrofolate reductase (DHFR) are routinely used to generate cell lines for the manufacture of therapeutic recombinant proteins. This design is utilized in the hope that the strong promoter can produce much higher levels of interested protein in comparison to the selection marker protein. The process for the selection of high-expressing clones is tedious and time consuming and often takes several months to one year, since only a small percentage of stable clones can express at such high levels.

Expression levels of an interested gene can often be appreciably increased by selection in methotrexate (MTX) (Urlab and Chasin, 1980; Kaufman and Sharp, 1982; Gasser et al., 1982; Page and Sydenham, 1991). MTX amplification procedures are often required to generate high expressing cells (over 20 pg/cell/day). However, this amplification is timing-consuming and often causes the instability demonstrated in expression levels of the cell lines. Recently glutamine synthetase (GS) selection systems were used to create high expression cell lines (over 20 pg protein/cell/day) in three months. It is believed that the GS systems provide more stringent selection pressure. Concomitantly, only a few resistant colonies can survive, with the relatively high percentage of the resistant colonies being high-expressing cell clones. However, these high-producing cell lines are often unstable in the absence of L-methionine DL-sulfoximine (MSX) (Cockett et al., 1990).

The alternate design disclosed in the present invention involves the use of a strong promoter to express both an interested gene and selection marker gene, while ensuring that the expression of the selection marker gene is much lower in comparison to expression of the interested protein. In this case, an internal ribosome entry site (IRES) is positioned between the interested gene and the downstream selection marker gene. Although using an attenuated IRES could reduce the expression of the selection marker gene and increase the expression level of the interested protein, the expression of the selection marker gene in this type of vector still resulted in not high enough stringent selection pressure and generated too many low-expressing clones. Besides, the mRNA containing both the interested gene and selection marker gene is much longer than that of the original form (i.e. promoter drives target protein coding region only, without IRES plus selection marker coding region), and affects cells' capacity to maximally produce the interested protein, due to low efficiency of mRNA translation.

Lucas et al. (1996) reported an expression vector design which produces both selection marker and interested protein from a single primary transcript via differential splicing. They showed that most of the primary transcripts were spliced to generate the mature form of mRNA to express the interested protein without any selection marker while small portions of the primary transcripts (immature mRNAs) escaped the splicing process and expressed only the selection marker (without interested gene). This vector has been used to generate several stable cell lines with high expression levels for a variety of recombinant proteins in stable CHO cell lines using dihydrofolate reductase (DHFR). These results suggest that higher ratios of mRNA encoding an interested protein to mRNA encoding a selection marker protein is desired in order to increase selection pressure (to have fewer clones survive the selection process) and at the same time increase the possibility for high expression (of interested protein) in cells that survive the selection process.

More recently, McGrew reported that an expression vector, in which an internal polyadenylation signal was inserted between a DNA sequence encoding a protein of interest and a DNA sequence encoding a selection marker, allows a single promoter to generate both monocistronic messages and bicistronic messages (U.S. Pat. No. 6,632,637). The principle of this selection approach is based on using the “leaky mRNA” to express the selection marker. Although the polyadenylation signal works as a transcriptional unit to terminate mRNA, small portions of the transcriptional units may bypass the polyadenylation signal to produce the longer RNA (leaky mRNA). There are several advantages of this selection approach. First, a single promoter drives both the interested gene and selection marker. Second, selection pressure may increase. McGrew demonstrated that cells transfected with the alternate polyadenylation vector had about 8 times as much IL-4R specific mRNA as the control, and the amount of DHFR was reduced 3.5-fold relative to the control. The transfected cells were amplified by a variety of the concentration of MTX (50, 100, 150 and 200 nM) and resulted in the increase of the expression level of IL-4. McGrew suggested that the efficiency of translation of the selection marker gene could be manipulated by altering the sequence of the IRES at or near the junction of the IRES adjacent to the selection marker gene. The reduction of the translation of marker gene therefore further increases the selection pressure and only allowed the clones of cells with higher promoter activity to survive. As a result, the higher promoter activity in these selected clones enhances the expression of the interested gene.

It is known that some of the polyadenylation signals (such as the Poly+(A) signal in retroviruses) are insufficient for transcriptional termination (leaky) and resulted in read-through, producing a longer mRNA. By doing this, retroviruses use the leaky transcriptional unit to activate and express cellular oncogenes from the host genome. In the case of retroviruses, leaky transcripts account for only 1-2% or less of total mRNA transcripts produced. If, per day, a single cell produces a recombinant protein of over 20 pg, the transcripts of the interested gene would account for only about 2-10% of the total mRNA in the cell. If read-through events happened at a rate of just 1% of the total transcripts of mRNA produced for the target protein, based on McGrew's invention, there would be a huge number of leaky mRNAs producing an undesirably high level of selection marker. Without a further reduction in the read-through events of the alternate Poly+(A) vector, this type of vector would likely prove unusable for the selection and generation of high-expression cell lines. Therefore, there is a need to find a way to reduce the expression of leaky mRNA further, by enhancing the blockage of the read-through of the single Poly+(A) signal to a minimum but yet can still allow the cell to survive under selection pressure.

### SUMMARY OF THE INVENTION

In one embodiment of the invention, there is provided an expression vector comprising a DNA sequence encoding a first protein, operably linked to a DNA sequence encoding a second protein, wherein a DNA sequence encoding at least two or more polyadenylation signal sequences is inserted between the DNA encoding the first protein of interest and the DNA encoding the second protein. In one embodiment, the second protein is a selection marker. Generally known selection markers include, but are not limited to, dihydrofolate reductase (DHFR), antibiotic resistance marker, and glutamine synthetase.

The internal polyadenylation DNA fragment contains at least two separate polyadenylation signals. In one embodiment, the first polyadenylation signal is a bovine growth hormone polyadenylation signal (BGHpA) which is linked to another polyadenylation signal or signals such as viral polyadenylation signals (SV40pA and TKpA, for example), cellular polyadenylation signals (e.g. bovine growth hormone and β-globin), or other artificial polyadenylation signals, or a DNA sequence which has similar function to polyadenylation signals and works or enhances transcription termination.

In one embodiment, the expression vector may further comprise an internal ribosome entry site (IRES) sequence between the DNA encoding the first protein, and the DNA encoding the second protein (selection marker gene), operably linked to both and downstream to the internal multi-polyadenylation sites. In one embodiment, the IRES is a mutant IRES such as a mutated IRES of EMCV so as to eliminate the potential promoter initiation site (transcriptional starting point).

The expression vector may further comprise a selection marker DNA sequence which is located downstream of IRES. In one embodiment, additional ATG sequences which are or are not in the same reading frame are positioned upstream of the selection marker ATG. For example, additional stop codons for translation which are or are not in the same reading frame are positioned downstream of IRES but upstream of the selection marker gene. In this way, the selection marker gene in the bicistronic configuration would be less efficient for translation.

In another embodiment of the invention, there is provided a set of vectors for recombinant antibody expression. For example, a vector may comprise two transcription units. The first transcription unit comprises sequentially a promoter DNA, a monoclonal antibody (mAb) light chain DNA, internal multi-polyadenylation sites, IRES DNA, selection marker gene DNA and a single polyadenylation site. A second transcriptional unit comprises a promoter DNA, a mAb heavy chain DNA and single- or multi-polyadenylation site DNA sequences. The IRES sequence is downstream of the internal multi-polyadenylation site, or mRNA splice donor and acceptor sites substantially as described by Lucas et al. (1996) operably linked to the internal multi-polyadenylation site and the DNA encoding the second protein/selection marker. An expression-augmenting sequence element may also be included upstream of the cloning site, operably linked thereto.

The present invention also provides host cells transfected with the expression vectors disclosed herein, yielding stable pools of transfected cells. Accordingly, another embodiment of the invention provides a transfected host cell; yet another embodiment provides a stable pool of cells transfected with the inventive expression vector. Also provided are cell lines cloned from pools of the transfected cells. In one embodiment, host cells are mammalian cells. In another embodiment, the host cells are CHO cells.

The present invention also provides a method for obtaining a recombinant protein, comprising transfecting a host cell with the expression vectors disclosed herein, culturing the transfected host cell under conditions promoting expression of the protein, and recovering the protein. The McGrew patent (U.S. Pat. No. 6,632,637) did not mention that the internal multi-polyadenylation signals could be used to increase selection pressure. In comparison with the use of a single polyadenylation signal, multi-polyadenylation signals provide a powerful approach to reduce the level of leaky mRNA in order to further enhance the selection pressure. In an usual application, transfected host cell lines are selected with two selection steps, the first to select for cells expressing the dominant amplifiable marker, and the second step for high expression levels and/or amplification of the marker gene as well as the gene of interest. A commonly used selection or amplification agent is methotrexate, an inhibitor of DHFR that has been shown to cause amplification of endogenous DHFR genes and transfected DHFR sequences. In one embodiment of the invention, the selection or amplification with MTX is not required. The host cells (e.g. CHO DG44) are transfected with DHFR selection vector and a high producing line can be directly selected by the HT-free medium without any MTX amplification.

To summarize, with a one promoter configuration (bicistronic), McGrew's one polyadenylation signal still allows too many read-throughs, especially in the case where the promoter activity happened to be very high. No stringent selection can be applied. With the present vector, dramatically reduced read-throughs result in fewer copies of mRNA for the selection marker, therefore stringent selection can help to obtain only the clones with high promoter activity. Moreover, because of the highly-stringent selection, the surviving clones already express the protein of interest at high levels, without the need to use MTX for amplification.

### BRIEF DESCRIPTION OF THE DRAWINGS

FIG. 1 shows one embodiment of vector design with polyadenylation signals inserted before IRES in the one promoter-two coding sequences vector.

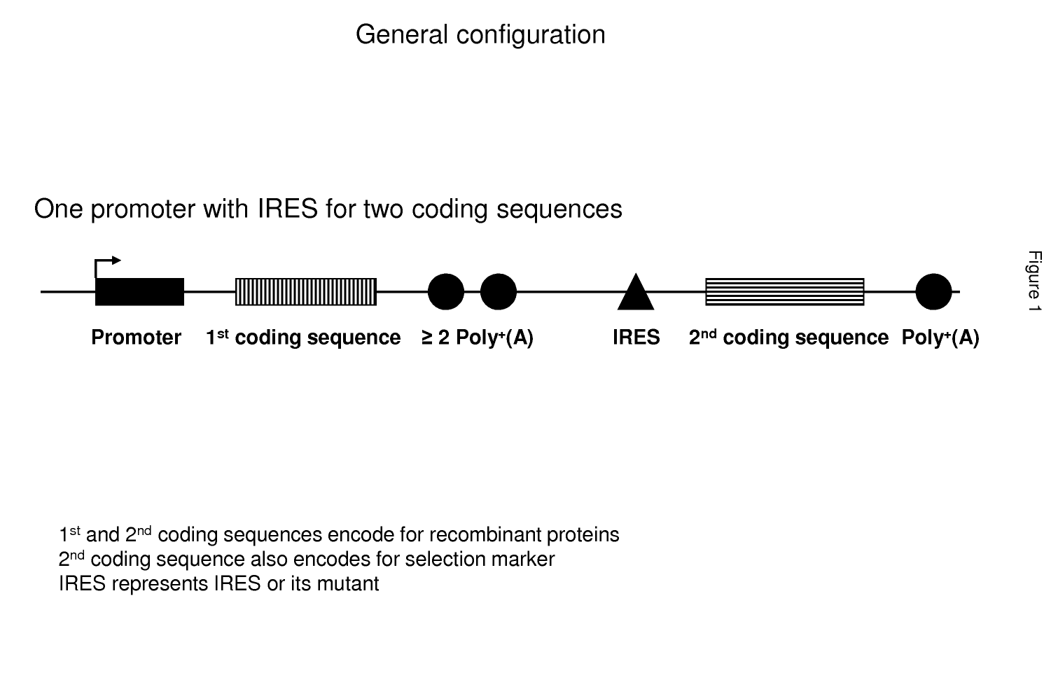

FIG. 2 shows another embodiment of vector design with recombinant monoclonal antibody expression and with DHFR as selection marker.

### DETAILED DESCRIPTION OF THE INVENTION

In one embodiment, the present invention discloses an eukaryotic expression vector with more than one sequential polyadenylation signal sequences [Poly+(A)] or one or more [Poly+(A)] signals plus any DNA fragment which is known to help reduce read-through (named enhanced termination unit), between two coding sequences (two peptide products) operably linked to one promoter positioned before the first coding sequence [bicistronic, i.e. promoter-1^{st} coding sequence-multiple Poly+(A)-IRES-2nd coding sequence], wherein the 2^{nd} coding sequence is for any selection markers, including, but not limited to dihydrofolate reductase (DHFR), antibiotic resistance markers (such as neomycin, puromycin, basticidin, hygromycin and zeocin resistance), and glutamine synthetase (GS).

The following terms shall be used to describe the present invention. In the absence of a specific definition set forth herein, the terms used to describe the present invention shall be given their common meaning as understood by those of ordinary skill in the art.

As used herein, the expression MTX refers to Methotrexate.

As used herein, the expression GS refers to glutamine synthetase.

As used herein, the expression pA or poly+(A) refers to polyadenylation signal.

As used herein, the expression pcd refers to pg per cell per day.

As used herein, the expression Her refers to herceptin monoclonal antibody.

As used herein, the expression L refers to monoclonal antibody light chain.

As used herein, the expression H refers to monoclonal antibody heavy chain.

As used herein, the expression DH refers to DHFR.

As used herein, the expression Sy refers to synthetic.

The present invention provides an eukaryotic expression vector comprising a first promoter operatively linked to a first coding sequence and an enhanced termination unit that comprises two or more polyadenylation signal sequence, followed by an internal ribosome entry site (IRES) and a second coding sequence. In one embodiment, the first coding sequence codes for a monoclonal antibody peptide. In another embodiment, the second coding sequence codes for a selection marker. Examples of selection markers include, but are not limited to, dihydrofolate reductase (DHFR), an antibiotic resistance marker, or glutamine synthetase. In general, the antibiotic resistance marker can be neomycin, puromycin, blasticidin, hygromycin or zeocin. In one embodiment, the expression vector has the sequence of SEQ ID NO:1.

In one embodiment, the polyadenylation signal sequences can be virus polyadenylation signal, cellular polyadenylation signal, a modified viral or cellular polyadenylation signal. For example, the polyadenylation signal sequences may have the sequence of nucleotides 1472-1783, 1941-2335, 3369-3673, or 5854-6160 of SEQ ID NO:1. Polyadenylation signal sequences are generally well-known in the art; representative examples include, but are not limited to, GenBank Accession Nos. M14147, V00111, M12890, D14486, D00683, and D00684. Furthermore, there are numerous review articles on RNA 3′ end processing; for example, Neilson and Sandberg, Exp. Cell Res. 316:1357-64 (2010); Millevoi and Vagner, Nucleic Acids Res. 38:2757-74 (2010); Licatalosi and Darnell, Nat. Rev. Genet. 11:75-87 (2010). In another embodiment, the polyadenylation signal can be artificial polyadenylation signal. One of ordinary skill in the art would readily construct a number of artificial polyadenylation signals. For example, artificial polyadenylation signal may have the sequence of nucleotides 1784-1940 of SEQ ID NO:1.

In another embodiment, the one or more polyadenylation signal sequences are followed by DNA fragment that has the function of reducing read-through. DNA sequences that have the function of reducing read-through are generally known in the art.

In one embodiment, the internal ribosome entry site (IRES) is a mutated IRES. For example, the mutated IRES may have the sequence of nucleotides 2336-2804 of SEQ ID NO:1. One of ordinary skill in the art would readily mutate or modify an IRES sequence for the practice of the present invention. In another embodiment, the IRES is inserted between a polyadenylation signal sequence and the second coding sequence such that the IRES is operably linked to the second coding sequence, and additional ATG(s) or plus termination codon(s) are located upstream of the second coding sequence.

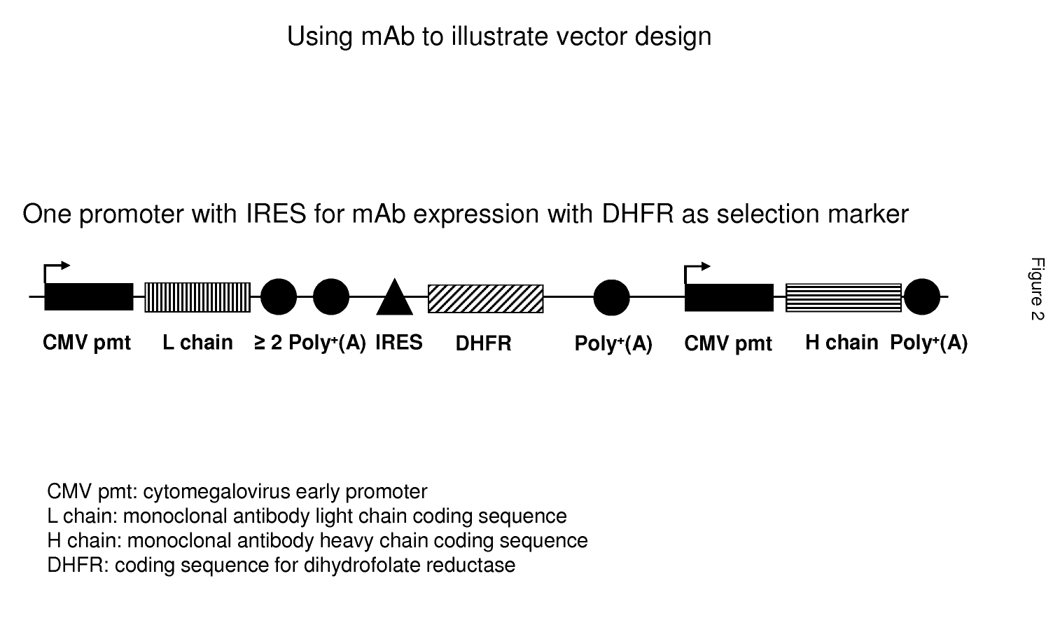

In another embodiment, the second coding sequence in the expression vector is followed by a second promoter operatively linked to a third coding sequence (see e.g. FIG. 2). In one embodiment, the third coding sequence encodes a monoclonal antibody peptide.

The present invention also provides cells or cell lines transfected with the expression vectors disclosed herein. In one embodiment, the cells are mammalian or eukaryotic cells. Examples of transfected cells include, but are not limited to, CHO-K1, CHO DG44, DXB11, NS0, BHK, Vero, Per C6 or HEK293 cells. In one embodiment, the cells are dihydrofolate reductase-deficient cells.

Throughout this application, various references or publications are cited. Disclosures of these references or publications in their entireties are hereby incorporated by reference into this application in order to more fully describe the state of the art to which this invention pertains.

It is to be noted that the transitional term “comprising”, which is synonymous with “including”, “containing” or “characterized by”, is inclusive or open-ended and does not exclude additional, un-recited elements or method steps.

### EXAMPLE 1

In this invention, the focus is on the effect of the read-through events on the selection marker gene expression. It is believed that 0.1-10% of the frequency of the read-through events produce 0.1-1% of the biscistronic transcriptional RNA in the total mRNA in the bicistronic configuration. McGrew (U.S. Pat. No. 6,632,637) reported an expression vector, in which one internal polyadenylation signal was inserted between a DNA encoding a protein of interest and a DNA encoding a selection marker, allowing a single promoter to generate both monocistronic messager RNA (normal product) and bicistronic messanger RNA (read-through product). The cells transfected with the alternate polyadenylation vector had about 8 times as much IL-4R specific messanger RNA (protein of interest) as the control, and the amount of DHFR was reduced 3.5-fold relative to the control, suggesting that 25% of the frequency of the read-through events happened and produced 25% of bicistronic mRNA containing the selection marker in the total mRNA. In the experiments of McGrew, the overall selection pressure appears to have remained relatively low, as demonstrated by the high number of read-through events. It is necessary to reduce the read-through events, decrease the production of the selection marker mRNA and increase the ratio of the interested protein mRNA to the selection marker mRNA by using more potent pA signals, more pA signals or plus other DNA fragments which are known to enhance pA termination function.

Several expression vectors containing different pA signals (SV40 late pA, bovine growth hormone pA and synthetic pA) with green fluorescent protein (GFP) as a reporter gene were constructed. If read-through events happen, longer bicistronic mRNA sequences will be generated and GFP will be expressed through IRES. These expression vectors were transfected into the 293T cells using Lipofectin 2000 (Invitrogen). To quantify the fluorescent signal of the transfected cells, fluorescence activated cell sorting (FACS) analysis was performed 48 hour after the transfection. The read-through events were quantified in comparison with that of the control vector. The table below shows the fluorescent signal of the transfected cells and ratio of the reduction of signal in comparison with that of the cells transfected by the vector with no internal pA signal. The data indicates that the frequency of the read-through events in different pA signals is different and is in a range between 1-6%, which is consistent with a previous report (U.S. Pat. No. 6,632,637). The bovine growth hormone (BGH) pA has the least read-through among the three pA signals tested. The data show that one bovine growth hormone pA signal in combination with three synthetic pA signals dramatically decreases the GFP level by 1175 fold, with the fluorescent signal levels dropping down almost to the background level (720 v 680). These data clearly demonstrated that the multi-internal pA signals can significantly reduce the read-through events, whereas a single internal pA signal is not sufficient.

| | GFP | |
|---|---|---|
| vector | mean | n-fold reduction |
| pCMV-L/Her | 680 | Baseline control |
| pCMV-L/Her-IRES-GFP | 47,311 | No pA control |
| pCMV-L/Her-SV40pA-GFP | 2,265 | 30 |
| pCMV-L/Her-SypA-GFP | 3,623 | 16 |
| pCMV-L/Her-BGHpA-GFP | 1,580 | 52 |
| pCMV-L/Her-BGHpA-pA-GFP | 1,107 | 110 |
| pCMV-L/Her-BGHpA-3pAs- | 720 | 1175 |
| GFP | | |

To obtain highly producing mammalian cell lines, large numbers of clones often need to be screened. This is largely due to low selection stringency, creating many, but low protein-producing clones. To overcome this problem and improve the efficiency of the selection process for high-producing clones, an expression vector system which can confer upon the transfected cells a means by which to withstand a very high stringent selection process needs to be developed so that only a few high level protein-producing clones will survive. It is possible that the multi-internal pA signals can generate more stringent selection pressure since the selection marker mRNA derived from the bicistronic mRNA could be dramatically reduced in comparison with a system utilizing a single internal pA signal.

To test the hypothesis, the GFP gene of the single or multi-pA vector was replaced with the DHFR gene. These vectors were designated as pCMV-Her-SV40pA-DHFR, pCMV-Her-BHGpA-DHFR, pCMV-Her-BHGpA-SypA-DHFR and pCMV-Her-BGHpA-3SypA-DHFR. Those vectors were transfected into CHO DG44 cells using the Lipofectamine 2000 system. After 48 hours of transfection, Herceptin (protein of interest) expression levels were measured by ELISA and 10 million transfected cells were placed into 10 plates of 96-well plate (10,000 cells/well) and grown in the selection medium. After 20 days of selection, the number of resistant colonies was determined under the microscope. Resistant colonies were grown in culture in the 96-well plate. Individual colony cultures from the original 96-well plate were replated in a second 96-well plate, and further separated in the 96-well plate into two cultures for analysis: one culture for analysis of the expression level using the terminal culture, and another culture for measuring cell growth of the individual colony. After 14 days of culture, most of those clones died because of lack of nutrition. The expression levels of Herceptin in these clones in the terminal culture condition were determined by ELISA Expression levels of 2.5 μg/ml in the culture medium were considered to be positive, whereas 50 μg/ml was considered to be high. Table 2 shows the number of resistant clones, positive and high expressing clones. These data showed that the number of the resistant clones dropped by increasing in the number of the internal pA signals. The number of resistant clones derived from the four internal pA vectors was reduced by 3 fold in comparison with that of the single internal pA vector (136 v 412), suggesting that the multi-pA vectors convene in the increase of the selection pressure.

Importantly, ELISA data show that 5% of these resistant clones (7/136) derived from a four-internal pAs vector (pCMV-Her-BGHpA-3SypAs-DHFR) expressed Herceptin over 50 μg/ml while only 1% of resistant clones (4/412) derived from a single-internal pA vector (pCMV-Her-BGHpA-DH) reached a level of Herceptin of 50 μg/ml, indicating that a multi-pA vector increased the chance or percentage for obtaining high-expressing clones. There is a trend that more high-expressing clones were obtained with the increase of Poly+(A) signals used in the expression vectors.

| | Num. of | | |
|---|---|---|---|
| Vector | clones | Pos. clones | High Exp. Clones |
| pCMV-Her-SV40pA-DHFR | 528 | 79(15%) | 2 |
| pCMV-Her-BGHpA-DHFR | 412 | 74(18%) | 4 |
| pCMV-Her-BGHpA-SypA- | 253 | 70(28%) | 4 |
| DHFR | | | |
| pCMV-Her-BGHpA- | 136 | 34(25%) | 7 |
| 3SypAs-DHFR | | | |

The ratio of antibody mRNA (short mRNA) to the long leaky mRNA containing the selection marker in the cells transfected with the four internal pA vector is assumed to be much higher than that with the single pA vector, after selection. This is in agreement with the previous data that the four pA vector decreased the GFP levels up to 20 fold in comparison with that of the single pA vector. It is also postulated that the four pA vector may not only increase the selection pressure, but also upregulate the promoter activity in order to produce enough DHFR to support cell survival. Because of this, the expression of the target protein was increased enormously by increased ratio of expression of the target protein to the selection marker, and by high selection pressure-mediated promoter activity up-regulation. This notion is supported by two sets of data collected separately. With McGrew's single pA vector, no high expression clone over 20 pg/cell/day was obtained from 10 million transfected cells after culture in selection medium without MTX-mediated amplification, whereas several clones using the present vector (multi-pA) survived. These data suggest that, in order to achieve that high level expression in the multi-pA clones, the promoter activity has to be pushed higher, likely due to enhanced selection stringency, or amplification had already occurred during the culture in the selection medium. However, when the vector copy number in the genome was quantified with quantitative polymerase chain reaction (PCR), the same range (1-10 copies per cell) of integrated vectors was found. Therefore, taking the data together, all the evidence confirms that the multi-pA vector not only increased the selection pressure by reducing the expression of selection marker through enhanced termination of bicistronic readthrough, but also further causes promoter activity increase as the result of selection pressure (negative feedback) until reaching balance in order to survive. This is fundamentally different from the single pA vector, whereas the increased expression of interested gene as reported in McGrew's patent was due to the selected group of higher expression clones resistant to relatively higher selection pressure, comparing to a selected cell population resistant to lower selection pressure.

This novel selected system allows us to manipulate the selection pressure in two ways: to control the amount of read-through bicisitronic mRNA using multiple pA signals (double or triple pA signals) and to change the translation efficiency of the bicisitronic mRNA using an attenuated IRES. First the first 136 bps of IRES region (which is not essential for IRES function) was deleted. Second the stop codon was inserted into the first initiating starting codon region (ATG GCG TAA ATG, SEQ ID NO:2) or the reading frame was shifted (ATG GCG GAT G) which may reduce the efficiency of the bicisitronic mRNA for translation (to express the DHFR) i.e. increase selection pressure. Also there are two TATA box like-motifs within the IRES region (81, 331). These motifs may serve as the promoter function to transcribe the mRNA to express the selection marker gene, independent of the bicisitronic mRNA. The TATAA motif at position 331 was mutated into TAAAA motif. This IRES containing the multiple mutations was called m2S-IRES. This mutant IRES was inserted between the synthetic pA signal and DHFR gene in pCMV-Her-BGHpA-3SypAs-DH and named as pCMV-Her-4pAs-DH (containing ATG GTT) or pCMV-Her-4pAs-2SDH (containing ATG GCG TAA ATG GTT, SEQ ID NO:3) or pCMV-Her-4pAs-2aaDH (containing ATG GCG G ATG GTT, SEQ ID NO:4). 10 million transfected cells derived from these vectors were placed into 10 96-well plates (10,000 cells/well) and grown in the selection medium. After 20 days of selection, the number of resistant cells per well was counted under a microscope. These resistant clones were divided into two groups: one for the analysis of the expression level using the terminal culture and the other for growing up the cells. After 14 days of culture, the expression level of these clones in the terminal culture condition was determined by ELISA. The data show that there were 98 resistant clones from pCMV-Her-4pAs-DH, while pCMV-Her-4pAs-2SDH generated 21 colonies. pCMV-Her-4pAs-2aa-DHFR produced no resistant clones, indicating that the multi-pA in combination with the attenuated IRES caused increase in selection pressure, resulting in dramatic decrease in the number of resistant clones or even no resistant clones. ELISA data show that the number of high expression clones derived from the attenuated IRES vector (pCMV-Her-4pAs-2SDHFR) was significantly diminished in comparison with that of the multi-pA vector alone (pCMV-Her- 4pAs-DHFR)(2 vs. 9). This data suggest that these expression clones may fail to survive in the selection medium because the selection pressure to the cells transfected with these attenuated IRES vector was too high. Therefore, with the inventive design, maximum selection pressure can be achieved. In one experiment, it was observed that four colonies produce over 1 μg/ml of antibody. Although both the resistant number and positive colonies (over 1 μg/ml) reduce significantly, one colony (identified as 373-6) expressed very high level (23 μg/ml). The single cell production of the 373-6 cell line is 20.4 pg/day/cell without any MTX amplification. This version of pA vector significantly reduced the frequency of resistant colonies (7 fold). These data suggest that this inventive vector could significantly increase in the selection pressure to reduce the amount of the screened resistant colonies. It indicates that high antibody-producing DG44 cell line can be generated in the selection culture medium without any MTX amplification using this novel vector system.

The inventive vectors and methods will also be useful in developing multicistronic vectors. Multicistronic expression vectors allow the coordinated expression of two or more genes (see, for example, Fussenegger et al., 1997). Inserting a polyadenylation site after a first cistron would result in high level expression of the first cistron and lower level expression of any following cistrons. Potential applications of this technology would be to facilitate expression of large amounts of a therapeutic protein (or other, desired recombinant proteins) and lower amounts of other proteins such as selection markers, transcription factors, enzymes involved in protein folding, and other proteins that regulate cell metabolism and expression.

In another embodiment, the polyadenylation site is inserted after the second or third (or subsequent) cistron. This would allow high expression of the first two (or three or more) cistrons, followed by lower expression of the cistron following the internal polyadenylation site. This embodiment will find use, for example, in recombinant antibody synthesis where the heavy and light chains are synthesized independently at high levels. A tricistronic vector is constructed with the heavy and light chains encoded by the first two cistrons. The polyadenlylation site is inserted following the second cistron allowing high level expression of the first two cistrons. The selectable marker is expressed from the third cistron (i.e., after the polyadenylation site) and would be expressed at lower levels.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### REFERENCES

Andersen, D. C., and Krummen, L. 2002. Recombinant protein expression for therapeutic applications Curr. Opin. Biotechnol., 13:117-123.

Cockett, M. I., Bebbington, C. R. and Yarranton, G. T. 1990. High level expression of tissue inhibitor of metalloproteinases in Chinese hamster ovary cells using glutamine synthetase gene amplification. BioTechnology, 8:662-667

Fussenegger et al., Biotechnol Prog 13:733; 1997.

Gasser, C. S., Simonsen, C. S., Schilling, J. W. and Schmike, R. T. 1982. Proc. Natl Acad. Sci. USA, 79:6522-6526.

Kaufman, R. J. and Sharp, P., J. 1982. Mol. Biol, 159:601-621.

Levinson, A. D. and Goeddel, D. C. 1990. Methods Enzymol., 185:485-487.

Licatalosi and Darnell. 2010. RNA processing and its regulation: global insights into biological networks. Nat. Rev. Genet. 11:75-87.

Lucas.B. K., Giere.L M., DeMarco. R. A, Shen.A, Chisholm.V Crowley C. W. 1996. High-level production of recombinant proteins in CHO cells using a dicistronic DHFR intron expression vector. Nucleic Acids Research, 24: 1774-1779.

McGrew, J. T. Vectors and methods for recombinant protein expression U.S. Pat. No. 6,632,637.

Millevoi and Vagner. 2010. Molecular mechanisms of eukaryotic pre-mRNA 3′ end processing regulation. Nucleic Acids Res. 38:2757-74.

Neilson and Sandberg, 2010. Heterogeneity in mammalian RNA 3′ end formation. Exp. Cell Res. 316:1357-64.

Page, M. J. and Sydenham, M. A. 1991. High level expression of the humanized monoclonal antibody CAMPATH-1H in Chinese hamster ovary cells, BioTechnology, 9:64-68.

Urlab, G. and Chasin, L. A. 1980. Isolation of Chinese hamster ovary cell mutant deficient in dihydrofolate reductase activity., Proc. Natl Acad. Sci. USA, 77:4216-4220.

Wurm, F. M., 2004. Production of Recombinant protein therapeutics in cultivated mammalian cells Nature Biotechnol., 22: 1393-1398.

1. An eukaryotic expression vector comprising a first promoter operatively linked to a first coding sequence and an enhanced termination unit that comprises two or more polyadenylation signal sequence, followed by an internal ribosome entry site (IRES) and a second coding sequence.

2. The expression vector of claim 1, wherein the second coding sequence codes for a selection marker.

3. The expression vector of claim 2, wherein the selection marker is dihydrofolate reductase (DHFR), an antibiotic resistance marker, or glutamine synthetase.

4. The expression vector of claim 3, wherein the antibiotic resistance marker is neomycin, puromycin, blasticidin, hygromycin or zeocin.

5. The expression vector of claim 1, wherein the first coding sequence encodes a monoclonal antibody peptide.

6. The expression vector of claim 1, wherein the polyadenylation signal sequences are selected from the group consisting of virus polyadenylation signal, cellular polyadenylation signal, a modified viral polyadenylation signal, a modified cellular polyadenylation signal, and artificial polyadenylation signal.

7. The expression vector of claim 1, wherein the one or more polyadenylation signal sequences are followed by DNA fragment that has the function of reducing read-through.

8. The expression vector of claim 1, wherein the vector has the sequence of SEQ ID NO:1.

9. The expression vector of claim 1, wherein the internal ribosome entry site (IRES) is a mutated IRES.

10. The expression vector of claim 1, wherein the IRES is inserted between a polyadenylation signal sequence and the second coding sequence such that the IRES is operably linked to the second coding sequence, and additional ATG(s) or plus termination codon(s) are located upstream of the second coding sequence.

11. The expression vector of claim 1, wherein the second coding sequence is followed by a second promoter operatively linked to a third coding sequence.

12. The expression vector of claim 11, wherein the third coding sequence encodes a monoclonal antibody peptide.

13. Cells or cell lines transfected with the expression vector of claim 1.

14. The cells or cell lines of claim 13, wherein the cells are mammalian or eukaryotic cells.

15. The cells or cell lines of claim 13, wherein the cells are selected from the group consisting of CHO-K1, CHO DG44, DXB11, NSO, BHK, Vero, Per C6 and HEK293 cells.

16. The cells or cell lines of claim 13, wherein the cells are dihydrofolate reductase deficient cells.


SEQUENCE LISTING (4 sequences)

SEQ ID NO: 1 - DNA, 6160 bp, Artificial Sequence
  Vector Sequence
aattctaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacaccccttggctcttatgcatgaattaagacagactgttcctttcctgggtcttttctgcaggcaccggatcctagcgctaccggtcgccaccatggagaccgacaccctgctgctgtgggtgctgctgctgtgggtgcccggcagcaccggcgacgacatccagatgacccagagcccctccagcctgtccgccagcgtgggcgacagggtgaccatcacctgccgggcctcccaggacgtgaacaccgccgtggcctggtaccagcagaagcccggcaaggcccccaagctgctgatctacagcgccagcttcctgtacagcggcgtgcccagcaggttctccggcagcaggagcggcaccgacttcaccctgaccatcagcagcctgcagcccgaggacttcgccacctactactgccagcagcactacaccaccccccccaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaactcgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaggcaatagcaggcatgctggggatgcggtgggctctatggcttttgaggcggaaagaaccagctggggctctagggggtatccccacgcgccctgtaagcctagaaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctactagaatgctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattactagtaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtttaagaaacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatggccacaaccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagtcgaggttcgctgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgtgaccccattggggccaatacgcccgcgtttcttccttttccccaccccaccccccaagttcgggtgaaggcccagggctcgcagccaccgtcggggcggcaggccctgccatagcatcccctatgctagaaaggaattctaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgaatggagacgccatccacgctgttttgacctccatagaagacaccgggaccgatccagcctccgcggccgggaacggtgcattggaacgcggattccccgtgccaagagtgacgtaagtaccgcctatagactctataggcacaccccttggctcttatgcatgaattaagacagactgttcctttcctgggtcttttctgcaggcaccggatcctagcgctaccggtcgccaccatggagaccgacaccctgctgctgtgggtgctgctgctgtgggtgcccggcagcaccggcgacgaggtgcagctggtggagagcggcggcggcctggtgcagcctggcggcagcctgaggctgagctgcgccgcctccggcttcaacatcaaggacacctacatccactgggtgcggcaggcccccggcaagggcctggagtgggtggccaggatctaccccaccaacggctacaccaggtacgccgacagcgtgaagggcaggttcaccatcagcgccgacaccagcaagaacaccgcctacctgcagatgaactccctgagggccgaggacaccgccgtgtactactgcagcagatggggcggcgacggcttatacgccatggactactggggccagggcaccctggtgaccacctccagcgccagcaccaaggggccctcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctatacagcaagctcaccgtggacaagagcaggtggcagcaggggaacgtcttctcatgctccgtgatgcatgaggctctgcacaaccactacacgcagaagagcctctccctgtctccgggtaaatgataactcgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaggcaatagcaggcatgctggggatgcggtgggctctatggcttttgaggcggaaagaaccagctggggctctagggggtatccccacgcgccctgtaa

SEQ ID NO: 2 - DNA, 12 bp, Artificial Sequence
  Vector Sequence
atggcgtaaatg

SEQ ID NO: 3 - DNA, 15 bp, Artificial Sequence
  Vector Sequence
atggcgtaaatggtt

SEQ ID NO: 4 - DNA, 13 bp, Artificial Sequence
  Vector Sequence
atggcggatggtt